(19) United States
(12) Patent Application Publication
(10) Pub. No.: US 2003/0212078 A1
(43) Pub. Date: Nov. 13, 2003

(54) Topical administration of pharmacological compositions for non-systemic treatment of pruritus

(76) Inventor: Gerald L. Klein, Glendale, CA (US)

Correspondence Address: Robert D Fish, Rutan & Tucker, 14th Floor, 611 Anton Boulevard, Costa Mesa, CA, 92626, US

(21) Appl. No.: 10/276,508

(22) PCT Filed: May 17, 2001
(86) PCT No.: PCT/US01/16100

(51) Int. Cl. A61K 31/542; A61K 31/495; A61K 31/453

(52) U.S. Cl. 514/255.04; 514/320; 514/224.8

(57) ABSTRACT

A pharmacological composition includes an anti-histaminic in spray formulation that reduces symptomatic skin itching of skin without substantially eliciting weight gain or sedation in the patient when the composition is sprayed onto the area of skin at a dosage of at least 10 mg/ml over a period of at least 10 applications. Particularly contemplated anti-histaminic include hydroxyzine and ketotifen.

### FIELD OF THE INVENTION

The field of the invention is topical treatment of pruritus.

### BACKGROUND OF THE INVENTION

Pruritus is a relatively common symptom of various allergic and non-allergic conditions, and there are numerous drugs and pharmacological compositions known in the art to treat such conditions (and thereby concomitantly to reduce the pruritus). For example, hydroxyzine hydrochloride (1-(p-chlorobenzhydryl) 4-[2-(2-hydroxyethoxy)-ethyl] piperazine dihydrochloride) exhibits significant efficacy in systemic management of pruritus due to allergic conditions such as chronic urticaria, atopic and contact dermatoses, and in systemic histamine-mediated pruritus.

Administration of Hydroxyzine for systemic treatment of pruritus typically employs oral delivery in tablets containing 10 mg, 25 mg, 50 mg, and 100 mg. Alternatively, Hydroxyzine can be ingested in liquid form as syrup at a hydroxyzine concentration of 10 mg/ml, or where oral administration is undesirable, hydroxyzine can be injected intramuscularly. In still another route of administration, Kanios et al. describe in U.S. Pat. No. 5,719,197 topical administration of Hydroxyzine in a substantially water insoluble formulation as a bioadhesive anti-histaminicum. Although Kanios' application of Hydroxyzine advantageously circumvents systemic administration, application of Hydroxyzine in a bioadhesive formulation is often undesirable, especially when applied over a relatively large area, or an area not covered by clothes (e.g., the face). Moreover, prolonged application of Hydroxyzine using Kanios' bioadhesive will at least to some extent result in systemic delivery of Hydroxyzine, which may lead to undesirable side effects, including drowsiness and tachycardia.

In another example, Ketotifen (4,9-Dihydro-4-(1-methyl-4-piperidinylidene)-10H-benzo-[4,5]cyclohepta[1,2-b]thiophene-10-one; U.S. Pat. No. 3,682,930 to Bourquin et al.) is employed in systemic treatment of pruritus. Such treatment has been described in various cases, for example in neurodermatitis [Effectiveness of ketotifen in the treatment of neurodermatitis in childhood; Kikindjanin, V., et al.; Dermatol. Monatsschr. 1990; 176(12); 741-744] and chronic urticaria [Treatment of chronic urticaria with Ketotifen; Egan, T. A. and Rallis T. M., Arch. Dermatol. 1997:133;147-149], both of which utilized a regimen of multiple oral doses of Ketotifen. Although oral administration of Ketotifen successfully relieved at least some of the symptoms, treatment was nevertheless limited to a systemic administration of Ketotifen in oral form.

Alternatively, Ketotifen may be topically administered in a cream preparation as described in Japanese Patent Application Laid-Open Gazette Nos. Sho. 51-32724, Sho. 51-142543, Sho. 62-164624, Hei. 1-102024 and Hei. 1-121218. However, the Ketotifen in such cream preparations is often instable (e.g., due to reaction with other components in the cream, poor release of Ketotifen from the preparation, or phase separation). To circumvent at least some of the problems associated with instability of Ketotifen in cream preparations, Nakagawa et al. describe a cream preparation that contains Ketotifen in a base comprising a diglycerol fatty acid ester and/or a sorbitan fatty acid ester and a polyvalent metal salt of a saturated or unsaturated fatty acid. Nakagawa's formulation advantageously increases the stability of Ketotifen, however, results in a formulation with relatively high percutaneous absorption. Consequently, Ketotifen is despite its topical application systemically administered in Nakagawa's formulation, thereby producing potentially various undesired systemic side effects (e.g., drowsiness and weight gain).

In further examples, various anti-histamines (e.g., phenothiazines, alkylamines or ethanolamines, piperidines, piperazines, diphenylhydramine, pheniramines, pyrilamine, promethazine and triprolidine, see also U.S. Pat. No. 2,567,245 describing pyridyl aliphatic amines with anti-histamine activity such as 3-(p-bromophenyl)-3-(2-pyridyl)-N,N-dimethylpropylamine and 3-(p-chlorophenyl)-3-(2-pyridyl)-N,N-dimethyl-propyl-amine [brompheniramine and chlorpheniramine], U.S. Pat. No. 2,712,023 describing pyridyl propenylamines with antihistamine activity such as (E)-1-(4-methylphenyl)-1-(2-pyridyl)-3-pyrrolidinoprop-1-ene [triprolidine]) may be employed for treatment of pruritus associated with allergic and non-allergic conditions. However, all or almost all of the anti-histamines tend to cause sedation or drowsiness when orally administered. Moreover, additional side effects include dryness of mouth, blurred vision and tachycardia, mostly due to varying degrees of anticholinergic activity.

Depending on the particular indication, some of the anti-histamines may be topically administered in a cream to reduce at least some of the problems associated with systemic side effects. However, and especially during a prolonged period of administration, various systemic side effects still persist. For example, topical administration of Adoxipin in a cream over several days will typically result in drowsiness and moderate to significant weight gain.

Alternatively, recently developed non-sedating antihistamines such as terfenadine, loratidine, or cetirizine may be orally administered to treat various allergic conditions. Although non-sedating antihistamines may advantageously be administered for treatment of systemic allergies, their effectiveness is frequently unsatisfactory for topical treatment of localized allergic conditions and itching.

Although various compositions and methods are known to treat allergic and non-allergic conditions, all or almost all of them suffer from one or more disadvantages. Therefore, there is still a need to provide improved compositions and methods to treat pruritus associated with thos conditions.

### SUMMARY OF THE INVENTION

The present invention is directed to methods and compositions of topical treatment of pruritus (which may or may not be associated with an allergic or a non-allergic condition) without generating one or more undesirable systemic side effects.

More particularly, contemplated methods include one step in which a pharmacological composition is provided that comprises a pharmacologically active compound having a desired topical effect and an undesired systemic effect. In another step, it is recognized that spraying the pharmacological composition to an area of skin of a patient elicits the desired effect without substantial generation of the undesired systemic effect in the patient, and in a further step, an area of skin of the patient in need of treatment with the pharmacological composition is identified. In a still further step, the pharmacological composition is sprayed onto the area of skin.

In one aspect of the inventive subject matter, the pharmacologically active compound comprises an anti-histaminic compound, preferably a piperazine (e.g., hydroxyzine) or tricyclic compound (e.g., Ketotifen or Doxepin). Further especially contemplated compounds include brompheniramine, chlorpheniramine, triprolidine, and phenothiazines.

In another aspect of the inventive subject matter, the pruritus is associated with an allergic condition and especially contemplated conditions include insect bites and stings, hives, atopic-, and contact dermatitis, and eczema, however, non-allergic pruritus, including uremic dermatitis, neurodermatitis, dry skin, and sunburn is also contemplated. Consequently, particularly contemplated desired topical effects comprise reduction of symptomatic itching. Especially contemplated undesired systemic effects comprise sedation, weight gain, and tachycardia.

In a further aspect of the inventive subject matter, a pharmacological composition includes an anti-histaminic in a spray formulation, wherein the composition reduces symptomatic itching on an area of skin of a patient without substantially eliciting at least one of a weight gain and a sedation in the patient when the composition is sprayed onto the area of skin at a dosage of at least 0.1 mg/ml, more preferably at least 1 mg/ml, and most preferably at least 10 mg/ml over a period of at least 10 applications. Particularly contemplated compositions include hydroxyzine, ketotifen, and agents selected from the group consisting of a phenothiazine, a piperazine, a piperidine, an alkyamine, an ethylenediamine, an ethanolamine, a tertiary amine tricyclic antidepressant, a norepinephrine-reuptake inhibitor, azatidine, and oxatomide.

Various objects, features, aspects and advantages of the present invention will become more apparent from the following detailed description of preferred embodiments of the invention.

### DETAILED DESCRIPTION

The inventors have surprisingly discovered that contemplated pharmacological agents and compositions can be topically applied onto a persons skin at a concentration sufficient to achieve a desired topical effect without eliciting an undesired systemic effect, when topical application comprises spraying the agents and compositions onto a persons skin.

Therefore, the inventors generally contemplate that in a method of treating a patient, a pharmacological composition is provided comprising a pharmacologically active compound having a desired topical effect and an undesired systemic effect. In a further step, it is recognized that spraying the pharmacological composition to an area of skin of a patient elicits the desired topical effect without substantial generation of the undesired systemic effect in the patient, and in a still further step, an area of skin of the patient in need of treatment with the pharmacological composition is identified, and the pharmacological composition is sprayed onto the area.

In a particularly preferred aspect, the method of treatment is a method of treating pruritus associated with an allergic condition (e.g., atopic dermatitis), and the pharmacological composition is a buffered isotonic solution of hydroxyzine, wherein hydroxyzine is present in a concentration of about 20-40 mg/ml, and wherein the buffered solution comprises an amphoteric buffer (e.g., TRIS) and optionally one or more preservatives and a coloring agent. An area in need of treatment with hydroxyzine is visually identified (e.g., by identifying areas of redness or dryness), and approximately 0.1 ml of the pharmacological composition is repeatedly sprayed (here: at least 10 times over the course of 2 days) onto the area in need using a conventional spray bottle, thereby reducing systemic itching of the area without substantial generation of sedation in the patient.

As used herein, the term “spraying” refers to any form of topical application of a substantially liquid pharmacological composition that disposes the composition in a plurality of droplets onto the surface of a patients skin. It should be particularly noted that “spraying” as used herein is limited to a surface deposition of the pharmacological composition, and particularly excludes any mechanism of significant transdermal and/or percutaneous systemic delivery (i.e., delivery of more than 10% of the pharmacological composition through the skin. The term “skin” as used herein is meant to include the dermis of the entire body surface, which may or may not include hair. In contrast, the surface of the eye or a mucous membrane is not considered the surface of a skin under the scope of this definition.

As further used herein, the term “desired topical effect” refers to any subjectively perceivable manifestation of application of contemplated pharmacological agents at the area to which the agent is applied that is due to the interaction of the pharmacological agent with its physiological target. For example, where the pharmacological agent is a histamine release inhibitor, the desired topical effect is a reduction in itching and/or heat perception. On the other hand, a cooling effect due to the formulation of the pharmacological agent (i.e., ethanolic solution) is not considered a desired topical effect, since the cooling is due to evaporation and not due to an interaction of the ethanol with a physiological target (e.g., a receptor, an enzyme, etc.).

Similarly, the term “undesired systemic effect” refers to any subjectively perceivable and/or objectively measurable manifestation of application of contemplated pharmacological agents in the patient that is due to the interaction of the pharmacological agent with its physiological target, wherein the manifestation is in an area other than the area to which the agent has been applied. For example, weight gain, drowsiness, or tachycardia are considered undesirable systemic effects, since the effect is in an area other that the area to which the agent has been applied.

The term “substantial generation of the undesired systemic effect” refers to a change of at least 2%, typically at least 3.5%, and more typically of at least 5% in the physiological parameter that is influenced by application of contemplated compounds. For example, where the undesired systemic effect is weight gain, a weight gain of 8 pounds in a person of 180 pounds is considered a substantial generation of the undesired systemic effect. Similarly, where the undesired systemic effect is tachycardia, an increase in a persons resting heart rate of about 5-10 beats per minute, is considered a substantial generation of the undesired systemic effect if the persons resting heart rate before application of contemplated compounds was 60 beats per minute. Where the undesired systemic effect is sedation, any subjectively perceived occurrence of drowsiness, reduction of anxiety, stress, irritability, or excitement associated with administration of contemplated compounds is considered a substantial generation of the undesired effect.

As still further used herein, the term “pruritus” refers to a cutaneous sensation that provokes a desire to rub or scratch the skin to obtain relief, and particularly refers to itching associated with and symptomatic of some other disease or abnormality. For example, the itching sensation associated with dry skin, an insect bite, or symptomatic itching of a contact dermatitis is referred to as pruritus under the scope of this definition. It should be especially noted, however, that the term “pruritus” as used herein does not refer to a disease or condition itself whose predominant symptom is itching. For example, pruritus does not refer to an allergic condition itself Consequently, contemplated methods of treating pruritus are particularly directed towards providing symptomatic relieve and may or may not treat an underlying condition that is associated with pruritus.

In an alternative aspect of the inventive subject matter, the pruritus need not necessarily be associated with atopic dermatitis, and various alternative allergic conditions are also contemplated, including an insect bite, an insect sting, hives, and contact dermatitis. Still further, it should be appreciated that not only allergic conditions, but also non-allergic conditions are contemplated, and particularly include sunburn, neurodermatitis, uremic dermatitis, dry skin, and eczema.

With respect to the pharmacologically active compound, it is contemplated that numerous compounds other than hydroxyzine are also suitable for use in conjunction with the teachings presented herein, and especially include anti-histaminic compounds. For example, appropriate pharmacologically active compound include various piperazines (e.g., Cetirizine, Cinnarizine, etc.), various tricyclic compounds with anti-histaminic activity (e.g. Ketotifen [4,9-dihydro-4-(1-methyl-4-piperidinylidene)-10H-benzocyclohepta[1,2-b]thiophen-10-one]), brompheniramine, chlorpheniramine, triprolidine, phenothiazine, etc. Furthermore, depending on the desired topical effect and occurrence of undesired systemic effect, suitable pharmacologically active compounds may also include antibiotic compounds, antifungal compounds, wound-healing compounds, compounds with cosmetic effect (e.g., anti-wrinkle, hydration, melanogenic compounds, etc.), and so forth.

Depending on the particular pharmacologically active compound, the pharmacological composition may vary considerably. However, it is generally contemplated that suitable compositions are liquids that can be sprayed from a conventional spray bottle (e.g., manually operated atomizer, pressurized atomizer, etc.). Especially preferred liquids are water-based liquid formulations or aqueous formulations comprising one or more cosolvents (e.g., ethanol, DMSO, glycerol, etc.), all of which may or may not be buffered with an organic or inorganic buffer. Furthermore, contemplated compositions may include additional ingredients, which may assist the contemplated pharmacologically active compound. There are numerous methods and compositions known in the art to produce suitable pharmacological compositions, and an exemplary collection of such methods and compositions is described in “Drug Formulation” by Racz, I., Akademiai Kiado, Budapest, 1989, AISN: 0471905178, in “Topical Drug Delivery Formulations” by David W. Osborne, Anton H. Amann (Editor), Marcel Dekker; ISBN: 082478183X or in “Transdermal and Topical Drug Delivery Systems” by Tapash K. Ghosh (Editor), William R. Pfister Editor), Su II Yum (Editor), Interpharm Press; ISBN: 1574910418.

While spraying of hydroxyzine to a selected area is particularly preferred, alternative formulations may also be in a cream-based forma. In a preferred cream-based formulation, the pharmacological composition comprising hydroxyzine is a C₁₈-fatty acid based cream containing 2% of hydroxyzine, titanium dioxide as a coloring agent, and a bisphenol as antimicrobial agent, which can be massaged into the affected area of skin at a dose of about 0.2 g of the composition per 10 cm² of affected skin. Alternatively, suitable formulations may also include formulations in a gel, mousse, ointment, or lotion (Compositions and methods for preparation of appropriate alternative formulations are well known to the art (supra).

With respect to the concentration of the pharmacologically active compound in the pharmacological composition, it should be appreciated that a particular concentration will predominantly depend on the type of pruritus (e.g., associated with an insect bite, or associated with contact dermatitis), and the overall size of the affected area to which the pharmacological composition is to be applied. For example, where smaller areas are treated and the itching is relatively strong, higher concentrations of contemplated pharmacologically active compound are contemplated, including concentrations of 4%-5% (corresponding to 40-50 mg/ml), more than 5% to 10% (corresponding to 50-100 mg/ml), and even more than 10% to 25% (corresponding to 100-250 mg/ml). On the other hand, where larger areas are treated, or where the itching is less severe, lower concentrations than 2% to 4% (corresponding to 20-40 mg/ml) are contemplated, including concentrations of less than 1% to 2% (corresponding to 10-20 mg/ml), and even concentrations of less than 1% (corresponding to less than 10 mg/ml).

With respect to additives, it should be appreciated that various additives may be included in the formulation, and suitable additives include pharmacologically active ingredients (e.g., pain relievers, wound healing promoters, or anti-scarring agents) as well as other additives, including coloring agents, preservatives, consistency regulators, sorbitol, cetyl alcohol, isopropyl alcohol, myristate, glyceryl stearate, PEG-100 stearate, petrolatum, benzyl alcohol, zinc acetate, or lactose. Further suitable ingredients may exhibit cooling properties, including volatile organic solvents, and volatile aromatic compounds.

In further alternative aspects of the inventive subject matter, it is contemplated that the step of identifying the skin area of a patient in need of treatment with contemplated compositions need not be restricted to visual identification by the patient. For example alternative methods of identifying the area include sensing (and visually confirming) the affected area. Identification may also include a person other than the patient, for example a physician, caregiver, or a family member. It should further be appreciated that the step of identification may include methods other than sensing and visual confirmation, including staining or thermoscanning. Although it is generally contemplated that the skin area is located on a human patient, it should also be appreciated that the skin area may be located on a non-mammal or mammal other than a human, including pets and live stock such as dogs, birds, rodents, horses, cows, pigs, etc.

In yet another aspect of the inventive subject matter, it is contemplated that pharmacological compositions may include various cosmetic preparations, and particularly contemplated cosmetic preparations are liquid and solid soaps, hair shampoo, hair conditioner, and cosmetic skin creams that reduce dryness and/or wrinkles of skin. There are many cosmetic preparations known in the art, and composition, consistency, and application purpose are not considered limiting to the inventive subject matter. For example, a commercially available hair shampoo or conditioner may be admixed with a 50 mg/ml hydroxyzine stock solution to obtain a modified shampoo having a final hydroxyzine concentration of 0.2% (by weight). In another example, where relatively high concentrations of hydroxyzine are desirable, hydroxyzine in solid form may be admixed to the shampoo or conditioner to obtain a final concentration of 5% (by weight). Similarly, conventional soaps may be admixed with appropriate amounts of hydroxyzine. With respect to the amounts of hydroxyzine in the cosmetic preparation the same considerations as described above apply.

Consequently, it is contemplated that a pharmacological composition in a spray formulation may comprise an anti-histaminic, wherein the composition reduces symptomatic itching on an area of skin of a patient without substantially eliciting at least one of a weight gain and a sedation in the patient when the composition is sprayed onto the area of skin at a dosage of at least 10 mg/ml over a period of at least 10, more preferably at least 20 applications. With respect to the anti-histaminic, itching, area of skin of the patient, and the spraying, the same considerations as described above apply.

In still further alternative aspects, contemplated pharmacological compositions may comprise a pharmacological agent in a spray formulation, wherein the pharmacological agent is selected from the group consisting of a phenothiazine, a piperazine, a piperidine, an alkyamine, an ethylenediamine, an ethanolamine, a tertiary amine tricyclic antidepressant, a norepinephrine-reuptake inhibitor, azatidine, and oxatomide. Contemplated compositions reduce symptomatic itching on an area of skin of a patient without substantially eliciting at least one of a weight gain and a sedation in the patient when the composition is sprayed onto the area of skin at a dosage of at least 10 mg/ml over a period of at least ten, more preferably at least twenty applications.

### EXAMPLES

In a small, uncontrolled and unblinded trial, two patients with pruritus were treated with a skin cream comprising 5% (by weight) hydroxyzine*HCl. The first patient was diagnosed with neurodermatitis (a non-allergic condition), while the second patient was diagnosed with an insect bite (an allergic condition). Remarkably, both patients showed a positive result (i.e., significant reduction in pruritus) after only 60 seconds.

In another small uncontrolled and unblinded trial, one group of patients with pruritus associated with atopic dermatitis was treated with a spray (aqueous buffered solution of 40 mg/ml hydroxyzine hydrochloride), which was topically applied to an area of the patients' skin affected by atopic dermatitis. The patients were instructed to spray about 0.1 ml (three pump actions of the manually operated spray bottle) of the composition onto an area of about 10 cm² affected by symptomatic itching for up to 5 applications per day for a period of two weeks. A second group of group of patients with pruritus associated with atopic dermatitis was treated with a cream (W/O emulsion containing 2% (wt) hydroxyzine hydrochloride), which was topically applied to an area of the patients' skin affected by atopic dermatitis. The patients were instructed to massage a small portion (about 0.2 gram) into an area of about 10 cm² affected by symptomatic itching for up to 5 applications per day for a period of two weeks.

Interestingly, almost all patients (13/15) in the second group experienced drowsiness and some of the patients (4/15) experienced transient tachycardia, while none of the patients in the first group experienced drowsiness or tachycardia. Even more interestingly, when hydroxyzine was replaced with cyproheptadine (using same concentrations and formulations for both groups) after 2 months of topical application of the spray and cream formulations in the respective groups, a significant portion (5/15) of the patients in the second group experienced weight gain of at least 4%, while there was no weight gain reported in the patients of the first group.

In a further physician-office based experiment with human volunteers, one group of 20 human volunteers and a second group of 5 human volunteers diagnosed with allergic and non-allergic dermatitis have been treated under the following protocol for a period of at least 30 days, and desired topical effects and undesired systemic effects are listed in Table 1.

Drug formulation: 0.5% (^{V}/v) of Ketotifen in aqueous buffered solution (pH 5.5, 20 mM Sodium-potassium phosphate, filter sterilized 0.2 μm); Application area: areas affected with allergic and non-allergic dermatitis between about 1 cm² and 25 cm²; Application and frequency: Approximately 50 μl/cm² per application, 3-5 times daily. Control: Oral Ketotifen as recommended.

TABLE 1

| | Ketotifen Spray | Oral Ketotifen |
|---|---|---|
| Symptomatic Relief | 18/20 | 4/5 |
| Weight Gain | 0/20 | 3/5 |
| Drowsiness | 0/20 | 4/5 |

While not whishing to be bound to any particular hypothesis, it is contemplated that the residence time of contemplated pharmacologically active compounds on the skin may have a prominent effect on the undesired systemic effect. Viewed from another perspective, it is contemplated that reducing the mode of drug presentation (i.e., burst presentation) may reduce the undesired systemic effect of the drug. In contrast, it is contemplated that increasing the time of drug presentation (i.e., sustained presentation) by lipophilic creams or application under occlusion patches may increase the undesired systemic effect. Still another contemplated aspects include delivery of the drug to produce a concentration spike and/or a relatively high rate of increase in the concentration of the drug in the body of the patient. Consequently, appropriate administrations need not be limited to spraying, but may also include dipping, application with a roller, brush, or other applicator. It is further contemplated that skin penetration enhancers may advantageously be included to further reduce the residence time, increase the concentration gradient or produce a concentration spike.

Thus, specific embodiments and applications of treating pruritus by topical administration of hydroxyzine have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of treating a patient, comprising:
providing a pharmacological composition comprising a pharmacologically active compound having a desired topical effect and an undesired systemic effect;
recognizing that spraying the pharmacological composition to an area of skin of a patient produces the desired topical effect without substantial generation of the undesired systemic effect in the patient;
identifying an area of skin of the patient in need of treatment with the pharmacological composition; and
spraying the pharmacological composition onto the area.

2. The method of claim 1 wherein the pharmacologically active compound comprises an anti-histaminic compound.

3. The method of claim 2 wherein the anti-histaminic compound comprises a piperazine.

4. The method of claim 3 wherein the piperazine is a 1-(p-chlorobenzhydryl) 4-[2-(2-hydroxyethoxy)-ethyl]piperazine dihydrochloride).

5. The method of claim 2 wherein the anti-histaminic compound comprises a tricyclic compound.

6. The method of claim 5 wherein the tricyclic compound comprises at least one of 4,9-dihydro-4-(1-methyl-4-piperidinylidene)-10H-benzocyclohepta[1,2-b]thiophen-10-one and doxepin.

7. The method of claim 2 wherein the anti-histaminic compound is selected from the group consisting of brompheniramine, chlorpheniramine, triprolidine, and a phenothiazine.

8. The method of claim 1 wherein the desired topical effect comprises reduction of symptomatic itching.

9. The method of claim 1 wherein the undesired systemic effect is selected from the group consisting of sedation, weight gain, and tachycardia.

10. The method of claim 1 wherein the area of skin is affected by symptomatic itching.

11. The method of claim 10 wherein the symptomatic itching is associated with an allergic condition.

12. The method of claim 11 wherein the allergic condition is selected from the group consisting of an insect bite, an insect sting, hives, atopic dermatitis, and contact dermatitis.

13. The method of claim 10 wherein the symptomatic itching is associated with at least one of a sunburn, a neurodermatitis, an uremic dermatitis, and an eczema.

14. A composition of matter comprising:
a pharmacological composition comprising an anti-histaminic in a spray formulation, wherein the composition reduces symptomatic itching on an area of skin of a patient without substantially eliciting at least one of a weight gain and a sedation in the patient when the composition is sprayed onto the area of skin at a dosage of at least 10 mg/ml over a period of at least ten applications.

15. The composition of claim 14 wherein the anti-histaminic is selected from the group consisting of a piperazine, a tricyclic compound, a pheniramine, and a phenothiazine.

16. The composition of claim 15 wherein the anti-histaminic comprises 1-(p-chlorobenzhydryl) 4-[2-(2-hydroxyethoxy)-ethyl]piperazine dihydrochloride) or 4,9-dihydro-4-(1-methyl-4-piperidinylidene)-10H-benzocyclohepta[1,2-b]thiophen-10-one.

17. The composition of claim 16 wherein the composition is sprayed onto the area of skin over a period of at least twenty applications.

18. A composition of matter comprising:
a pharmacological composition comprising a pharmacological agent in a spray formulation;
wherein the pharmacological agent is selected from the group consisting of a phenothiazine, a piperazine, a piperidine, an alkyamine, an ethylenediamine, an ethanolamine, a tertiary amine tricyclic antidepressant, a norepinephrine-reuptake inhibitor, azatidine, and oxatomide; and
wherein the composition reduces symptomatic itching on an area of skin of a patient without substantially eliciting at least one of a weight gain and a sedation in the patient when the composition is sprayed onto the area of skin at a dosage of at least 10 mg/ml over a period of at least ten applications.

19. The composition of claim 18 wherein the symptomatic itching is associated with an allergic condition selected from the group consisting of an insect bite, an insect sting, hives, atopic dermatitis, and contact dermatitis.

20. The composition of claim 18 wherein the composition is sprayed onto the area of skin over a period of at least twenty applications.
